(19) United States
(12) Patent Application Publication
(10) Pub. No.: US 2019/0332657 A1
(43) Pub. Date: Oct. 31, 2019

(54) AUTOMATED LINKING OF MEDIA DATA

(71) Applicant: Sorenson IP Holdings, LLC, Salt Lake City, UT (US)

(72) Inventors: Michael Jones, Farmington, UT (US); Adam Montero, Midvale, UT (US)

(21) Appl. No.: 15/963,872

(22) Filed: Apr. 26, 2018

(51) Int. Cl. G06F 17/24; G10L 15/22; G10L 15/08; G06K 9/00; G06F 17/27; G16H 80/00; G16H 30/20

(52) U.S. Cl. CPC G06F 17/24; G10L 15/22; G10L 15/08; G10L 2015/088; G06F 17/2765; G16H 80/00; G16H 30/20; G06K 9/00624

(57) ABSTRACT

Operations related to linking media data related to an information sharing session may include obtaining image media corresponding to the information sharing session and obtaining transcript data that includes a transcription of audio. The operations may further include generating image data that includes identification of objects depicted in the image media. In addition, the operations may include obtaining a keyword related to a topic of the information sharing session and identifying a transcript data segment of the transcript data based on the transcript data segment corresponding to the keyword. Moreover, the operations may include identifying an image data segment of the image data based on the image data segment corresponding to the keyword. The operations may also include inserting, in the transcript data segment, an image tag that indicates the related image of the image data segment.

### FIELD

The embodiments discussed herein are related to automated linking of media data.

### BACKGROUND

Information sharing sessions (e.g., in-person interactions, telephonic communication sessions, video communication sessions, presentations, lectures, etc.) may have different types of media associated therewith. For example, in some circumstances, media that corresponds to information sharing sessions may include audio media (e.g., audio recordings, audio streams) of audio (e.g., words spoken) of the information sharing sessions, audio media presented or shared during the information sharing sessions, image media (e.g., pictures, video recordings, video streams, etc.) of what occurs during the information sharing session, image media presented or shared during the information sharing sessions, textual media (e.g., text messages, documents, presentation materials, etc.) presented or shared during the information sharing sessions, or the like. Sometimes, records may be generated based on information sharing sessions and the corresponding media that may be associated with the information sharing sessions.

The subject matter claimed herein is not limited to embodiments that solve any disadvantages or that operate only in environments such as those described above. Rather, this background is only provided to illustrate one example technology area where some embodiments described herein may be practiced.

### SUMMARY

Operations related to linking media data related to an information sharing session may include obtaining transcript data that includes a transcription of audio of the information sharing session and obtaining image media corresponding to the information sharing session. The operations may further include generating image data that includes identification of objects depicted in the image media and that indicates which images included in the image media correspond to which objects. In addition, the operations may include obtaining a keyword related to a topic of the information sharing session and identifying a transcript data segment of the transcript data based on the transcript data segment including a related word of the transcription that have subject matter related to the keyword. Moreover, the operations may include identifying an image data segment of the image data based on the image data segment including a related image that depicts a related object that corresponds to the keyword. The operations may also include, in response to the transcript data segment and the image data segment both corresponding to the keyword, inserting, in the transcript data segment, an image tag that indicates the related image of the image data segment.

The objects and advantages of the embodiments will be realized and achieved at least by the elements, features, and combinations particularly pointed out in the claims. Both the foregoing general description and the following detailed description are given as examples and are explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1 illustrates an example environment related to linking of media that is related to an information sharing session;

FIG. 2 illustrates an example environment related to linking of media in an information sharing session between two parties;

FIG. 3 is a flowchart of an example method of linking media data related to an information sharing session;

FIG. 4 is a flowchart of another example method of linking media data related to an information sharing session;

FIG. 5 is a flowchart of an example method of determining follow-up data related to an information sharing session; and

FIG. 6 illustrates an example computing system that may be used to link media data and/or to generate follow-up data related to an information sharing session, all arranged according to one or more embodiments described in the present disclosure.

### DESCRIPTION OF EMBODIMENTS

Some embodiments of the present disclosure relate to the linking of media data related to information sharing sessions. For example, media data that corresponds to media associated with information sharing sessions (e.g., in-person interactions, telephonic communication sessions, video communication sessions, presentations, lectures, etc.) may be obtained or generated. The media data may include audio data of what is spoken during the information sharing sessions, audio data of audio media presented or shared during the information sharing sessions, image data of what occurs during the information sharing session, image data of image media presented or shared during the information sharing sessions, textual data of textual media presented or shared during the information sharing sessions, or the like.

According to one or more embodiments of the present disclosure, segments of the different media data types associated with an information sharing session may be automatically linked with each other by a computing system based on the segments corresponding to similar subject matter. The linking may improve a user's ability to review the information sharing session by allowing the user to more easily identify and access the portions of the media that are related to each other. The linking may also allow the user to more quickly identify and access each of the different portions of the media that are related to a particular subject.

Additionally or alternatively, according to some embodiments of the present disclosure, image data that may be associated with an information sharing session may be analyzed. Based on the analysis, follow-up data with respect to the information sharing session, such as additional information or questions that may be obtained regarding a topic or discussion of the information sharing session may be determined. The follow-up data may help improve the information sharing session by providing additional insights regarding the information sharing session.

In short, the present disclosure provides solutions to problems in artificial intelligence, networking, telecommunications, and other technologies to enhance records related to information sharing sessions. For instance, the records may be enhanced by improving the media data associated with the information sharing sessions through linking of the media data in a manner that improves the navigability of the records. Additionally or alternatively, the records may be enhanced with the determined follow-up data. Embodiments of the present disclosure are explained in further detail below with reference to the accompanying drawings.

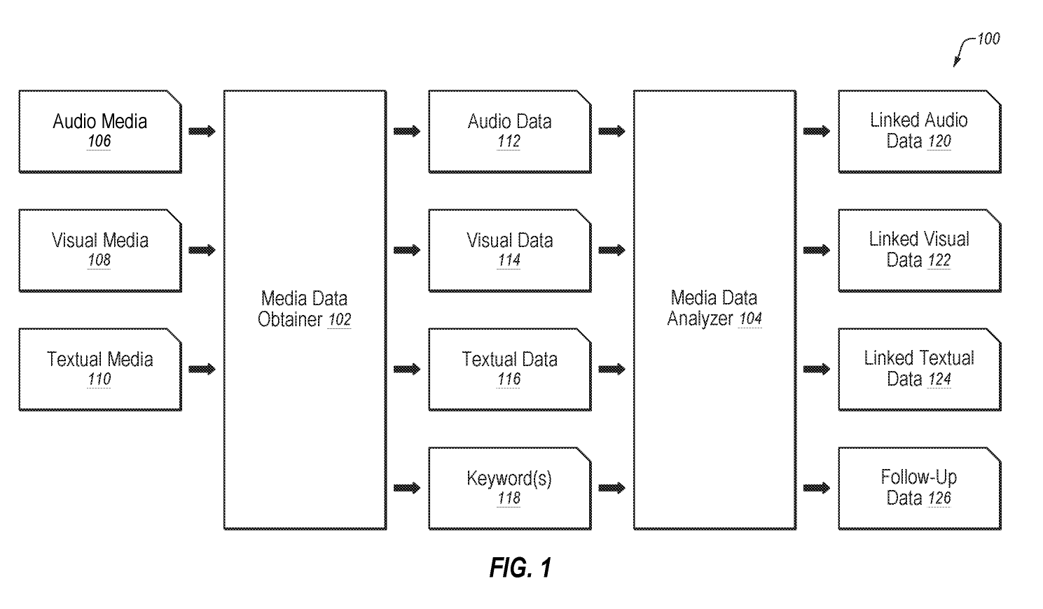

Turning to the figures, FIG. 1 illustrates an example environment 100 related to linking of media that is related to an information sharing session. The environment 100 may be arranged in accordance with at least one embodiment described in the present disclosure. The information sharing session may include any type of interaction or presentation during which information may be shared. For example, the information sharing session may be a live presentation, a recorded presentation, a conversation between two or more persons (e.g., in person, over a telephone call, over a video conference, etc.), a healthcare professional/patient interaction (e.g., in person, over a telephone call, over a video conference, etc.) or any other applicable presentation or interaction.

In some embodiments, the environment 100 may include a media data obtainer 102 configured to obtain one or more of: audio data 112 based on audio media 106; image data 114 based on image media 108; and textual data 116 based on textual media 110. Additionally or alternatively, the environment 100 may include a media data analyzer 104 configured to generate one or more of: linked audio data 120, linked image data 122, linked textual data 124, and follow-up data 126 based on one or more of: the audio data 112, the image data 114, the textual data 116, and one or more keywords 118.

In some embodiments, the audio media 106 may include any audio that may be part of or correspond to the information sharing session. For example, the audio media 106 may include an audio stream of audio of the information sharing session. For instance, the audio stream may be audio that may be communicated between devices during a telephone call, video conference, etc. In these or other embodiments, the audio stream may include the audio of a conversation or audio of a presentation as captured by a microphone. In these or other embodiments, the audio media 106 may include recorded audio of the information sharing session. Additionally or alternatively, the audio media 106 may include audio that is shared or presented during the information sharing session. For example, the audio media 106 may include recorded audio that is played during the information sharing session. In these or other embodiments, the audio media 106 may include one or more audio files of recorded audio. In the present disclosure, reference to “audio” may include audio in any format, such as a digital data format, an analog data format, or a soundwave format.

In some embodiments, the image media 108 may include any images or series of images that may be part of or correspond to the information sharing session. The images may be individual, still images such as pictures or sequential images captured as video. For example, the image media 108 may include a video stream of video of the information sharing session. For instance, the video stream may be video that may be communicated between devices during a video conference, etc. In these or other embodiments, the video stream may include video of a presentation as captured by a camera. In these or other embodiments, the image media 108 may include recorded video of the information sharing session or pictures of the information sharing session that may be captured.

Additionally or alternatively, the image media 108 may include images (e.g., pictures, video, etc.) that are captured, shared, or presented during the information sharing session. For example, the image media 108 may include recorded video that is played during the information sharing session or pictures that are presented during the information sharing session. Additionally or alternatively, the image media 108 may include recorded video or one or more pictures that are communicated between devices during the information sharing session (e.g., pictures or video sent during a telephone conversation). In these or other embodiments, the image media 108 may include images or video that are captured during the information sharing session. In these or other embodiments, the image media 108 may include one or more picture or video files. In the present disclosure, reference to “video” or “pictures” may include video or pictures in any format, such as a digital data format or an analog data format.

In some embodiments, the textual media 110 may include any media that may include text and that may be part of or correspond to the information sharing session. By way of example, the textual media 110 may include text configured in any suitable format that may be shared or presented during the information sharing session. For instance, the textual media 110 may include text messages, e-mail messages, social media posts, shared comments, online comments, documents (e.g., .pdf documents, word processing documents, pamphlets, paper hand-outs), slide-show presentations, sensor readings, statistics, etc. In these or other embodiments, the textual media 110 may include one or more files. In some instances, image media 108 may also be considered textual media 110. For example, images that may include text may be considered textual media 110 and/or image media 108. Additionally or alternatively, the textual media 110 may include other visual media. For example, in some instances, the textual media 110 may include charts or graphs in which the text may provide indications about the information represented by the charts or graphs. The distinctions and delineations included in the present disclosure are to help facilitate understanding and explanation and are not meant to mutually exclusive in all instances.

As indicated above, the media data obtainer 102 may be configured to obtain one or more of: the audio data 112 based on the audio media 106; the image data 114 based on the image media 108; and the textual data 116 based on the textual media 110. In some embodiments, the media data obtainer 102 may include computer readable instructions configured to enable a computing device to obtain the audio data 112, the image data 114, or the textual data 116, as described in the present disclosure. Additionally or alternatively, the media data obtainer 102 may be implemented using hardware including a processor, a microprocessor (e.g., to perform or control performance of one or more operations), a field-programmable gate array (FPGA), or an application-specific integrated circuit (ASIC). In the present disclosure, operations described as being performed by the media data obtainer 102 may include operations that the media data obtainer 102 may perform itself or direct a corresponding system to perform.

In some embodiments, the audio data 112 may include the audio media 106 such that the media data obtainer 102 may obtain the audio data 112 by obtaining the audio media 106. For example, in some embodiments, the audio media 106 may include an audio recording (e.g., an audio file) and the audio data 112 may include the audio recording.

Additionally or alternatively, the media data obtainer 102 may perform one or more operations with respect to the audio media 106 to generate the audio data 112. For example, as indicated above, in some instances, the audio media 106 may include an audio stream and the media data obtainer 102 may be configured to record the audio stream to generate an audio file of recorded audio that may be included in the audio data 112.

In these or other embodiments, the media data obtainer 102 may also be configured to obtain transcript data that may include a transcription of audio of the audio media 106. The transcript data may be included in the audio data 112 in some embodiments. The media data obtainer 102 may be configured to obtain the transcript data based on the audio media 106 according to any suitable technique or mechanism. For example, in some embodiments, the media data obtainer 102 may be configured to obtain the transcript data by communicating the audio media 106 to a transcription system separate from the media data obtainer 102. In these and other embodiments, the transcription system may generate the transcript data and communicate the transcript data to the media data obtainer 102. Alternatively or additionally, the media data obtainer 102 may be configured to obtain the transcript data by generating the transcript data. For example, in some embodiments, the media data obtainer 102 may be configured to generate the transcript data using one or more automatic speech recognition engines or techniques.

In these or other embodiments, the media data obtainer 102 may be configured to obtain audio timing data that corresponds to the audio media 106. In these or other embodiments, the media data obtainer 102 may be configured to include the audio timing data with the audio data 112. For example, the audio media 106 may include an audio recording of the information sharing session that includes audio timestamps that indicate when particular portions of audio occurred during the information sharing session. In these or other embodiments, the audio media 106 may include an audio stream and the media data obtainer 102 may be configured to generate timestamps for the corresponding audio of the audio stream. In some embodiments, the media data obtainer 102 may be configured to generate the audio timestamps as the audio stream is received. Additionally or alternatively, the media data obtainer 102 may be configured to generate an audio recording of the audio stream and generate the audio timestamps during or after generation of the corresponding audio recording. Additionally or alternatively, one or more of the audio timestamps may already be included in the received audio media 106 and may be included in the audio data 112.

In these or other embodiments, the audio timing data may include a time at which a corresponding audio recording of the audio media 106 may be captured, shared, or presented (e.g., played) during the information sharing session. For example, during the information sharing session an audio recording may be shared between multiple devices, captured by a device, or presented. In some embodiments, the media data obtainer 102 may be configured to generate one or more audio-sharing timestamps that indicate when the audio recording was captured, presented, or shared. In these or other embodiments, the media data obtainer 102 may be configured to identify when the audio recording was captured, presented, or shared, and may generate the audio-sharing timestamps based on the identification. In some embodiments, the media data obtainer 102 may be configured to identify when the audio recording was presented, captured, or shared, based on: a user indication; an analysis of words spoken during the information sharing session (e.g., by analyzing the transcript data) that indicate that the audio recording was captured, presented, or shared; reception of an indication that the audio recording was captured, presented, or shared; or in any other suitable manner.

In some embodiments, the audio timestamps or audio-sharing timestamps may include absolute times such as a time of day, a time of day and a date, etc. Additionally or alternatively, the audio timestamps or audio-sharing timestamps may include relative times such as a time from a beginning or a time until an ending of a corresponding information sharing session or some other defined time during the information sharing session.

In these or other embodiments, the media data obtainer 102 may be configured to generate transcript timing data with respect to the transcript data that may be included in the audio data. In some embodiments, the transcript timing data may indicate a point in time during the information sharing session that the words included in the corresponding transcription were spoken. For example, in some embodiments, the transcript timing data may be based on the timestamps in the audio timing data that may correspond to words included in the transcription.

In these or other embodiments, the transcript timing data may include transcript timestamps that each indicate a point in time during the information sharing session that corresponding words were spoken. In some embodiments, the transcript timestamps may include absolute times such as a time of day, a time of day and a date, etc. Additionally or alternatively, the transcript timestamps may include relative times such as a time from a beginning or a time until an ending of a corresponding information sharing session or some other defined time during the information sharing session.

In some embodiments, the image data 114 may include the image media 108 such that the media data obtainer 102 may obtain the image data 114 by obtaining the image media 108. For example, in some embodiments, the image media 108 may include a video recording (e.g., a video file) and the image data 114 may include the video recording. Additionally or alternatively, the image media 108 may include a picture (e.g., a picture file) and the image data 114 may include the picture.

Additionally or alternatively, the media data obtainer 102 may perform one or more operations with respect to the image media 108 to generate image data 114. For example, as indicated above, in some instances, the image media 108 may include a video stream and the media data obtainer 102 may be configured to record the video stream to generate a video file that includes recorded video that may be included in the image data 114.

In these or other embodiments, the media data obtainer 102 may be configured to process the images included in the image media 108. For example, the media data obtainer 102 may be configured to process the images by analyzing the images using one or more image recognition techniques. Additionally or alternatively, the media data obtainer 102 may be configured to identify one or more objects included in the images based on the image recognition. In these or other embodiments, an indication of the identified objects and an indication as to which images (e.g., pictures, video frames, etc.) the identified objects correspond may be included in the image data 114. The image recognition, object identification, and corresponding generation of indications of identified objects, and to which images the identified objects correspond may transform the image data 114 such that the image data 114 is searchable with respect to objects depicted in the images of the image data 114.

The media data obtainer 102 may be configured to employ any suitable technique to perform the image recognition. For example, the media data obtainer 102 may be configured to use image processing to identify characteristics of objects included in the images and may identify the objects based on the identified characteristics. In these or other embodiments, the media data obtainer 102 may use machine-learning as part of the object identification.

In these or other embodiments, the media data obtainer 102 may be configured to perform text identification with respect to the images included in the image media 108. Additionally or alternatively, the media data obtainer 102 may be configured to identify one or more words included in the images based on the text identification. In these or other embodiments, an indication of the identified words and an indication as to which images (e.g., pictures, video frames, etc.) the identified words correspond may be generated and included in the image data 114.

The text identification and corresponding indications of identified words and to which images the identified words correspond may also help transform the image data 114 such that the image data 114 is searchable with respect to objects depicted in the images of the image data 114. For example, in some instances, the objects depicted in the images may include tags that may indicate what the objects are. For instance, with respect to a healthcare professional/patient interaction, an object depicted in a particular image may be a medicine container. The media data obtainer 102 may accordingly identify text of the medicine container that may help identify information about the medicine container such as that the medicine container is a medicine container, a type of medicine contained by the medicine container, dosage information, a doctor who prescribed the medication, a pharmacy that provided the medication, a prescription date, a number of refills, etc.

The media data obtainer 102 may be configured to employ any suitable technique to perform the text identification. For example, the media data obtainer 102 may be configured to identify the text using optical character recognition.

In these or other embodiments, the media data obtainer 102 may be configured to obtain image timing data that corresponds to the image media 108 and to include the image timing data as part of the image data 114. For example, the image media 108 may include a video recording of the information sharing session that includes video timestamps that indicate when particular events related to particular portions of the video recording occurred during the information sharing session. In these or other embodiments, the image media 108 may include a video stream and the media data obtainer 102 may be configured to generate video timestamps for the corresponding frames of the video stream. In some embodiments, the media data obtainer 102 may be configured to generate the video timestamps as the video stream is received. Additionally or alternatively, the media data obtainer 102 may be configured to generate a video recording of the video stream and generate the video timestamps during or after generation of the corresponding video recording.

In these or other embodiments, the image media 108 may include one or more pictures of the information sharing session that each include a picture timestamp that indicates when particular events related to the corresponding pictures occurred during the information sharing session. In these or other embodiments, the media data obtainer 102 may be configured to generate the picture timestamps as the corresponding pictures are received by the media data obtainer 102 as part of the image media 108. Additionally or alternatively, one or more of the picture timestamps may already be included in the received image media 108 and may be included in the image data 114.

In these or other embodiments, the image timing data may include a time at which a corresponding image or video of the image media 108 may be captured, shared, or presented during the information sharing session. For example, during the information sharing session, a video may be captured, presented (e.g., played), or shared between multiple devices or a picture may be captured, presented, or shared between multiple devices. In some embodiments, the media data obtainer 102 may be configured to generate one or more image-sharing timestamps that indicate when the corresponding images were captured, presented (e.g., as video), or shared. In some embodiments, the media data obtainer 102 may be configured to identify when the images were captured, presented, or shared based on: a user indication; an analysis of words spoken during the information sharing session (e.g., by analyzing the transcript data) that indicate that the images were captured, presented, or shared; reception of an indication that the images were captured, presented, or shared; or in any other suitable manner.

In some embodiments, the image timestamps (e.g., video timestamps, picture timestamps, image-sharing timestamps) may include absolute times such as a time of day, a time of day and a date, etc. Additionally or alternatively, the image timestamps may include relative times such as a time from a beginning or a time until an ending of a corresponding information sharing session.

In some embodiments, the textual data 116 may include the textual media 110 such that the media data obtainer 102 may obtain the textual data 116 by obtaining the textual media 110. For example, in some embodiments, the textual media 110 may include a text message and the textual data 116 may include the text message. Additionally or alternatively, the textual media 110 may include a word processing document and the textual data 116 may include the word processing document.

Additionally or alternatively, the media data obtainer 102 may perform one or more operations with respect to the textual media 110 to generate textual data 116. For example, in some instances, the textual media 110 may include a particular document that is in PDF format or is formatted as an image of the particular document. In these or other embodiments, the media data obtainer 102 may be configured to perform character recognition, such as optical character recognition, with respect to the PDF or image to identify words and characters included in the particular document. Based on the character recognition, the media data obtainer 102 may be configured to generate a version of the particular document that is in a searchable format and that may be included in the textual data 116.

As another example, in some instances, the textual media 110 may include data of more than one format. For instance, the textual media 110 may include text messages, e-mails, word processing documents, PDF documents, images of documents, presentation documents, etc. In some embodiments, the media data obtainer 102 may be configured to convert the textual media 110 into one particular format and the converted textual media 110 may be included in the textual data 116.

As another example, in some instances, the textual media 110 may include visual media such as charts or graphs. In some embodiments, the media data obtainer 102 may be configured to identify text in the charts or graphs using any suitable technique. In these or other embodiments, the media data obtainer 102 may be configured to include the identified text in association with the corresponding visual media in the textual data 116.

In these or other embodiments, the media data obtainer 102 may be configured to obtain textual timing data that corresponds to the textual media 110 and to include the textual timing data with the textual data 116. For example, the textual timing data may include a time at which particular textual media of the textual media 110 may be captured, shared, or presented during the information sharing session. For instance, during the information sharing session, a text message may be shared between multiple devices. In some embodiments, the media data obtainer 102 may be configured to generate one or more text timestamps that indicate when the corresponding textual media was shared. As another example, in some instances (e.g., during a healthcare professional/patient interaction) telemetric data (e.g., heart rate readings, blood pressure readings, electrocardiogram (EKG) readings, etc.) may be included in the textual media 110 communicated during part of the information sharing session. In these or other embodiments, the media data obtainer 102 may be configured to generate timestamps as to when the telemetric data was captured or shared. In some embodiments, the media data obtainer 102 may be configured to identify when the corresponding textual media was captured, presented, or shared based on a user indication, an analysis of words spoken during the information sharing session (e.g., by analyzing the transcript data) that indicate that the corresponding textual media was captured, presented, or shared, or in any other suitable manner.

In some embodiments, the text timestamps may include absolute times such as a time of day, a time of day and a date, etc. Additionally or alternatively, the text timestamps may include relative times such as a time from a beginning or a time until an ending of a corresponding information sharing session.

As indicated above, the media data analyzer 104 may be configured to generate one or more of: linked audio data 120, linked image data 122, linked textual data 124, and follow-up data 126 based on one or more of: the audio data 112, the image data 114, the textual data 116, and the one or more keywords 118. In some embodiments, the media data analyzer 104 may include computer readable instructions configured to enable a computing device to obtain the linked audio data 120, the linked image data 122, the linked textual data 124, and/or the follow-up data 126, as described in the present disclosure. Additionally or alternatively, the media data analyzer 104 may be implemented using hardware including a processor, a microprocessor (e.g., to perform or control performance of one or more operations), a field-programmable gate array (FPGA), or an application-specific integrated circuit (ASIC). In the present disclosure, operations described as being performed by the media data analyzer 104 may include operations that the media data analyzer 104 may perform itself or direct a corresponding system to perform.

In general, the media data analyzer 104 may be configured to generate the linked audio data 120, the linked image data 122, and the linked textual data 124 by linking data segments of the audio data 112, the image data 114, and/or the textual data 116 that are related to each other. In some embodiments, the media data analyzer 104 may be configured to determine that the data segments are related to each other based on the data segments having related subject matter. In the present disclosure, general use of the terms “data segment” or “data segments” may refer to data segments of the audio data 112, the image data 114, and/or the textual data 116.

For example, in some embodiments, the media data analyzer 104 may be configured to obtain one or more keywords 118. The keywords 118 may include one or more words that may correspond to a topic of the information sharing session. For example, the keywords 118 may include the subject matter of a presentation or a purpose of an interaction. In these or other embodiments, the keywords 118 may include words that may be related to the subject matter or purpose. For example, in some embodiments, the keywords 118 may include one or more words that are commonly associated with a particular topic of a presentation. As another example, a particular interaction may be a particular healthcare professional/patient interaction. In these or other instances, the keywords 118 may include words that may commonly be part of a medical interaction in general or that may commonly be part of the type of medical interaction of the specific healthcare professional/patient interaction such as a particular purpose of the medical interaction.

In these or other embodiments, the keywords 118 may be based on profile data of one or more participants in the information sharing session. For example, in some embodiments, the information sharing session may be the particular healthcare professional/patient interaction and the keywords 118 may be based on profile data of the patient. The profile data may include information about the patient, such as demographic information, including name, age, sex, address, etc., among other demographic data. The profile data may further include health related information about the patient. For example, the health related information may include the height, weight, medical allergies, past telemetric readings, current medical conditions, etc., among other health related information. In some embodiments, the profile data may include transcriptions of conversations between the patient and the healthcare professional. In these or other embodiments, the keywords 118 may be based on profile data of the healthcare professional that may include credentials, an expertise level, a specialty, education, etc. of the healthcare professional.

As another example, the information sharing session may be a presentation. In these or other embodiments, the keywords 118 may be based on information about the receivers of the presentation such as education level, field of expertise, level of exposure to the topic of the presentation, etc. Additionally or alternatively, the keywords 118 may be based on the credentials, expertise, specialty, etc. of the presenter of the presentation.

In some embodiments, the media data analyzer 104 may be configured to obtain the keywords 118 by receiving the keywords 118. For example, the media data analyzer 104 may receive the keywords 118 as user input in some embodiments. Additionally or alternatively, the media data analyzer 104 may receive the keywords 118 from another system, apparatus, device, or program that may have previously determined the keywords 118.

In these or other embodiments, the media data analyzer 104 may be configured to determine the keywords 118. For example, in some embodiments, the media data analyzer 104 may be configured to perform topic analysis operations with respect to the audio data 112, the image data 114, and/or the textual data 116 to identify subject matter of the audio data 112, the image data 114, and/or the textual data 116. Based on the topic analysis, the media data analyzer 104 may be configured to generate one or more of the keywords 118 that correspond to the identified topics.

As another example, in some embodiments, the media data analyzer 104 may be configured to identify participants in the information sharing session (e.g., based on user input, information obtained from systems managing the information sharing session and/or devices participating in the information sharing session, and/or an analysis of the audio data 112, the image data 114, and/or the textual data 116). The media data analyzer 104 may be configured to acquire information about the identified participants and to generate the keywords 118 based on the acquired information. For instance, in some embodiments, the media data analyzer 104 may be configured to access patient and/or professional profiles related to the patient and healthcare professional involved in the particular healthcare professional/patient interaction. Based on information included in the records, the media data analyzer 104 may be configured to obtain one or more of the keywords 118. In some embodiments, the media data analyzer 104 may be configured to identify certain terms or types of terms included in the records and to use such terms as one or more of the keywords 118. In these or other embodiments, the media data analyzer 104 may be configured to identify related terms that may be related to the terms included in the records and may be configured to use one or more of the related terms as one or more of the keywords 118.

In some embodiments, the media data analyzer 104 may be configured to search the audio data 112, the image data 114, and/or the textual data 116 based on the keywords 118. For example, the media data analyzer 104 may be configured to search for the keywords 118 or for terms related to the keywords in the audio data 112, the image data 114, and/or the textual data 116. For instance, the media data analyzer 104 may be configured to search through the transcript data of the audio data 112 and may identify one or more transcript data segments of the transcript data that include related words that may correspond to the subject matter of the keywords 118.

As another example, the media data analyzer 104 may be configured to search through the image data 114 and may identify one or more image data segments of the image data 114 that include related images that depict objects that correspond to the keywords 118. In some embodiments, the image data segments may each be a portion of the image data 114 that corresponds to one or more images. For example, a particular image data segment may include one or more individual image files that may be included in the image data 114. In some embodiments, the media data analyzer 104 may identify which image data segments include which objects based on the object identification described above. As another example, the media data analyzer 104 may be configured to search through the textual data 116 and may identify one or more textual data segments of the textual data 116 that include one or more related portions of the textual media that have subject matter related to the keywords 118.

The media data analyzer 104 may be configured to link data segments that correspond to one particular keyword 118. For example, the media data analyzer 104 may identify a particular transcript data segment, a particular image data segment, and a particular textual data segment that each correspond to the particular keyword 118. In response to the particular transcript data segment and the particular image data segment corresponding to the particular keyword 118, the media data analyzer 104 may be configured to link the particular image data segment with the particular transcript data segment. In these or other embodiments, the media data analyzer 104 may be configured to link the particular image data segment with other audio data segments that may correspond to the transcript data segment. For example, the particular image data segment may be linked with another particular audio data segment that includes the audio that corresponds to the particular transcript data segment. In these or other embodiments, the particular textual data segment may be linked with the particular image data segment, the particular transcript data segment, and/or the particular transcript data segment in response to the particular textual data segment corresponding to the particular keyword 118.

Additionally or alternatively, the media data analyzer 104 may be configured to determine that particular data segments (e.g., a particular audio data segment, a particular image data segment, and/or a particular textual data segment) are related to each other based on the particular data segments corresponding to similar points in time of the information sharing session. For example, in some embodiments, the media data analyzer 104 may be configured to analyze audio timing data, image timing data, and/or textual timing data that may be included in the audio data 112, the image data 114, or the textual data 116, respectively, to identify audio data segments, image data segments, and textual data segments that may correspond to similar points in time of the information sharing session. In some embodiments, the media data analyzer 104 may be configured to identify data segments that have timing data that corresponds to points in time during the information sharing session that are within a particular timeframe with respect to each other. The size of the particular timeframe may vary according to different implementations. By way of example, the size of the particular timeframe may be between 1 second and 1 minute. Additionally or alternatively, the size of the particular timeframe may be based on an amount of time associated with speaking a sentence, a paragraph, or an amount of time from when a first participant of the information sharing sessions finishes speaking, a second participant then begins speaking and finishes speaking, and the first participant begins speaking again.

In some embodiments, the media data analyzer 104 may be configured to use timestamps included in the audio timing data, the image timing data, and/or the textual timing data to identify which data segments may correspond to each other based on timing. For example, the image timing data of the image data 114 may include a particular image-sharing timestamp that may indicate a time at which particular image media included in a particular image data segment of the image data 114 may be presented during the information sharing session. Further, audio timing data of the audio data 112 may include transcript timing data that may indicate when words of a corresponding transcription occurred during the information sharing session. The media data analyzer 104 may be configured to identify a particular transcript data segment that corresponds to the particular image-sharing timestamp based on the particular image-sharing timestamp and the transcript timing data indicating that the particular transcript data segment corresponds to a similar point in time as the particular image-sharing timestamp (e.g., based on the transcript timing data indicating that the particular transcript data segment corresponds to a point in time that is within a particular timeframe with respect to the particular image-sharing timestamp). In these or other embodiments, the media data analyzer 104 may be configured to link the particular transcript data segment with the particular image data segment in response to the particular transcript data segment and the particular image data segment corresponding to similar points in time of the information sharing session.

In these or other embodiments, the media data analyzer 104 may be configured to use the timing data to make further linking determinations with respect to data segments that may be linked based on one or more of the keywords 118. For example, in some instances, the media data analyzer 104 may determine that a first transcript data segment and a second transcript data segment may both correspond to a particular keyword 118. Additionally, the media data analyzer 104 may determine that a particular image data segment may also correspond to the particular keyword 118. Additionally, based on the audio timing data and the image timing data, the media data analyzer 104 may make a first determination that the first transcript data segment and the particular image data segment correspond to points in time during the communication session that are within a particular timeframe with respect to each other—e.g., that the words of the first transcript data segment and the images of the particular image data segment were communicated during the information sharing session within the particular timeframe with respect to each other. Further, based on the audio timing data and the image timing data, the media data analyzer 104 may make a second determination that the second transcript data segment and the particular image data segment do not correspond to points in time during the communication session that are within the particular timeframe with respect to each other—e.g., that the words of the second transcript data segment and the images of the particular image data segment were not communicated during the information sharing session within the particular amount of time with respect to each other. In some embodiments, in response to the first determination and the second determination, the media data analyzer 104 may link the first transcript data segment with the particular image data segment but may not link the second transcript data segment with the particular image data segment. The linking of data segments based on both timing data and subject matter similarity may provide greater accuracy and/or granularity in identifying which data segments may be more relevant to other data segments.

In some embodiments, the media data analyzer 104 may be configured to link data segments based on a hierarchal categorization of the keywords 118 and/or of the subject matter of the data segments. For example, in some embodiments, a particular keyword 118 may correspond to a general category that may include sub-elements. For instance, a particular keyword 118 may be “medicine” and individual medications may be sub-elements of the particular keyword 118. In these or other embodiments, data segments that correspond to a sub-element may also be linked with data segments that correspond to the particular keyword 118 in general but may not necessarily be linked with data segments that correspond to other sub-elements.

For example, the media data analyzer 104 may identify a first transcript data segment that corresponds to “medicine” but not any medicine in particular and may identify a second transcript data segment that corresponds to a first particular medicine. Additionally, the media data analyzer 104 may identify a first image data segment that corresponds to “medicine” but not any medicine in particular and may identify a second image data segment that corresponds to a second particular medicine. In some embodiments, the media data analyzer 104 may be configured to link the first transcript data segment with the first image data segment and the second image data segment in response to the first transcript data segment, the first image data segment, and the second image data segment all falling under the general category of “medicine.” Similarly, the media data analyzer 104 may be configured to link the second transcript data segment with the first image data segment in response to the second transcript data segment and the first image data segment both falling under the general category of “medicine. In these or other embodiments, in some instances, the media data analyzer 104 may not link the second transcript data segment with the second image data segment because although the second transcript data segment and the second image data segment both correspond to medicine in general, they each correspond to different medicines. Alternatively, in some instances, the media data analyzer 104 may link the second transcript data segment with the second image data segment because both the second transcript data segment and the second image data segment both correspond to medicine in general even though they each correspond to different medicines.

Additionally or alternatively, the media data analyzer 104 may be configured to link data segments that are categorized under same categories in the present disclosure. For example, the media data analyzer 104 may be configured to link different transcript data segments that may correspond to one particular keyword 118. Additionally or alternatively, the media data analyzer 104 may be configured to link other audio data segments (e.g., audio data segments that include actual audio) that correspond to the particular keyword 118 with each other and/or with one or more of the transcript data segments that also correspond to the particular keyword 118. In these or other embodiments, the media data analyzer 104 may be configured to link data segments that are categorized under same categories in the present disclosure based on timing data indicating that the linked data segments correspond to points in time during the information sharing session that are within the particular amount of time with respect to each other.

In some embodiments, the media data analyzer 104 may be configured to generate the linked audio data 120, the linked image data 122, and the linked textual data 124 based on the linking of data segments. For example, the media data analyzer 104 may link a particular audio data segment, a particular image data segment, and a particular textual data segment. Additionally or alternatively, the media data analyzer 104 may be configured to generate a particular audio tag for the particular audio data segment, a particular image tag for the particular image data segment, and a particular textual tag for the particular textual data segment.

The particular audio tag may include an indication of the audio that may correspond to the particular audio data segment. The indication of the audio may include presentation of the audio, a selectable link that provides access to the particular audio data segment in response to selection, a reference to a filename that corresponds to the particular audio data segment, or any other applicable type of indication.

Additionally or alternatively, the particular audio data segment may include a particular transcript data segment. In these or other embodiments, the particular audio tag may include a particular transcript tag that may include an indication of one or more words that may be included in the particular transcript data segment. The indication of the one or more words may include presentation of the one or more words, a selectable link that provides access to the particular transcript data segment in response to selection, a reference to a filename that corresponds to the particular transcript data segment, or any other applicable type of indication.

The particular image tag may include an indication of the images that may correspond to the particular image data segment. The indication of the images may include presentation of the images (e.g., as still pictures or video depending on the type of image), a selectable link that provides access to the particular image data segment in response to selection, a reference to a filename that corresponds to the particular image data segment, or any other applicable type of indication.

The particular textual tag may include an indication of one or more portions of textual media that correspond to the particular textual data segment. The indication of the portions of the textual media may include presentation of the portions, a selectable link that provides access to the particular textual data segment in response to selection, a reference to a filename that corresponds to the particular textual data segment, or any other applicable type of indication.

In these or other embodiments, the media data analyzer 104 may be configured to generate the linked audio data 120, the linked image data 122, and the linked textual data 124 using the generated tags. For example, in some embodiments, the media data analyzer 104 may be configured to insert the particular image tag and/or the particular textual tag in the particular audio data segment to generate linked audio data 120. As indicated above, in some embodiment, the particular audio data segment may include the particular transcript data segment and the media data analyzer 104 may be configured to insert the particular image tag and/or the particular textual tag in the particular transcript data segment to generate linked audio data 120. In these or other embodiments, the media data analyzer 104 may be configured to insert, in the particular audio data segment, one or more other audio tags, image tags, and/or textual tags that may correspond to one or more other audio data segments, image data segments, and/or textual data segments, respectively, that may be linked with the particular audio data segment as part of the generation of the linked audio data 120.

Additionally or alternatively, the media data analyzer 104 may be configured to insert the particular audio tag and/or the particular textual tag in the particular image data segment to generate linked image data 122. In these or other embodiments, the media data analyzer 104 may be configured to insert, in the particular image data segment, one or more other audio tags, image tags, and/or textual tags that may correspond to one or more other audio data segments, image data segments, and/or textual data segments, respectively, that may be linked with the particular image data segment as part of the generation of the linked image data 122.

In these or other embodiments, the media data analyzer 104 may be configured to insert the particular audio tag and/or the particular image tag in the particular textual data segment to generate linked textual data 124. Additionally or alternatively, the media data analyzer 104 may be configured to insert, in the particular textual data segment, one or more other audio tags, image tags, and/or textual tags. The other audio tags, image tags, or textual tags may correspond to one or more other audio data segments, image data segments, and/or textual data segments, respectively. The other audio data segments, image data segments, and textual data segments that may be linked with the particular textual data segment as part of the generation of the linked textual data 124.

In these or other embodiments, the media data analyzer 104 may be configured to dynamically update the tagging of data segments in the linked data. For example, following the tagging of the particular audio data segment, the particular image data segment, and the particular textual data segment, a new data segment may be identified as corresponding to the same keyword 118 as the particular audio data segment, the particular image data segment, and the particular textual data segment. The new data segment may be an audio data segment, an image data segment, or a textual data segment. In some embodiments, the media data analyzer may be configured to update the tagging based on the new data segment such that a new tag that corresponds to the new data segment is inserted in the particular audio data segment, the particular image data segment, and/or the particular textual data segment. Additionally or alternatively, the media data analyzer 104 may be configured to update a reference associated with the particular audio tag, the particular image tag, and/or the particular textual tag with the new tag such that selection of one of the particular audio tag, the particular image tag, and/or the particular textual tag may also reference the new tag. Thus, all data segments that are tagged as being associated with a particular keyword 118 may be associated together and the selection of one of the particular audio tag, the particular image tag, the new tag, and the particular textual tag may reference the others of the particular audio tag, the particular image tag, the new tag, and the particular textual tag. Additionally or alternatively, the particular audio tag, the particular image tag, and/or the particular textual tag may be inserted in the new data segment. As such, the media data analyzer 104 may be configured to update one or more of the linked audio data 120, the linked image data 122, or the linked textual data 124 in a dynamic manner in response to identifying the new data segment.

In these or other embodiments, the media data analyzer 104 may be configured to generate follow-up data 126 based on the audio data 112, the image data 114, and/or the textual data 116. For example, in some instances, the audio data 112, the image data 114, and/or the textual data 116 may indicate information about the information sharing session that may be used to determine other information or questions that may be related to the subject matter of the information sharing session. The other information or questions may be identified and used as the follow-up data 126 in some embodiments.

For example, the information sharing session may be a healthcare professional/patient interaction in some instances. Additionally, particular image data 114 may include one or more particular images that may indicate an injury, a health condition (e.g., a skin condition, eye dilation, skin color, body fat percentage of the patient, etc.), a medicine container, etc. Based on the object and/or textual data that may be included in the image data 114, the media data analyzer 104 may be configured to determine follow-up data 126 based on the images that may be included in the image data 114 in which the follow-up data 126 may relate to the injury, health condition, medicine container, etc.

For instance, the object and/or text recognition included in the particular image data 114 may indicate that the particular images correspond to a particular type of injury or health condition. In some embodiments, the media data analyzer 104 may be configured to identify the particular injury or health condition based on the particular image data 114 and may generate, as follow-up data 126, additional questions or identify other information that may correspond to the particular type of injury or health condition. The questions or information may include questions or information about other symptoms or potential side effects, complications, or other health issues that may be associated with the injury or health condition that may not be identifiable from the corresponding images. In these or other embodiments, the questions or information may include questions or information about other injuries or health conditions that may be related to or mistaken with the identified injury or health condition. In some embodiments, the media data analyzer 104 may be configured to generate the additional questions or information based on a database of medical information that may be include information on injuries and health conditions.

As another example, in some instances, the object and/or text recognition included in the particular image data 114 may indicate that the particular images include a medicine container. In some embodiments, the media data analyzer 104 may be configured to identify, based on the particular image data 114, information about the medicine container including a corresponding medication, dosage information, refill information, prescribing doctor information, issuing pharmacy information, prescription date, amount of doses remaining, etc. In these or other embodiments, the media data analyzer 104 may be configured to generate as follow-up data 126, additional questions or identify other information that may be based on the analysis of the particular image data 114. For example, the media data analyzer 104 may be configured to estimate a number of pills left in the medicine container and correlate the number of pills with a date filled and dosage amount to determine whether the patient may be appropriately taking the medication. Such information may be included in the follow-up data 126.

As another example, the media data analyzer 104 may be configured to identify the specific medicine included in the medicine container of the particular image based on text included in the particular image data 114 identified through textual identification as described above. In some embodiments, the media data analyzer 104 may be configured to identify contraindications (e.g., other drugs, alcohol, procedures, etc.) that may not be recommended in conjunction with the identified specific medicine. The contraindications may be included in the follow-up data 126 as follow-up questions or information in some embodiments.

As another example, the textual data 116 may include telemetric data about the patient. In some embodiments, the media data analyzer 104 may be configured to generate follow-up data 126 that may be related to or based on the telemetric data. For instance, the telemetric data may include information indicating that the patient has high blood pressure and the follow-up data 126 may include questions regarding the patient's diet, exercise, medication, etc. with respect to blood pressure.

In some embodiments, the media data analyzer 104 may be configured to provide the follow-up data 126 to one or more participants in the information sharing session. In these or other embodiments, the media data analyzer 104 may be configured to provide the follow-up data 126 during or after the information sharing session. For example, during a healthcare professional/patient interaction, the media data analyzer 104 may be configured to provide the follow-up data 126 to the healthcare professional such that the healthcare professional may incorporate the follow-up data 126 in the interaction. The media data analyzer 104 may be configured to provide the follow-up data 126 by communicating the follow-up data 126 to a device of the recipient of the follow-up data 126, directing that the follow-up information be displayed on the applicable device, or via any other suitable mechanism or technique.

Modifications, additions, or omissions may be made to the environment 100 without departing from the scope of the present disclosure. For example, in some embodiments, the media data obtainer 102 and the media data analyzer 104 may be included on a same system or device. Additionally or alternatively, the media data obtainer 102 and the media data analyzer 104 may be included on separate systems or devices. Further, the description of the operations of the media data obtainer 102 and the media data analyzer 104 are to aid in the understanding of the present disclosure and the delineation between the media data obtainer 102 and the media data analyzer 104 is not meant to be limiting. For example, in some implementations, a first system or device may perform one or more, but not necessarily all, of the operations described with respect to both the media data obtainer 102 and the media data analyzer 104. In these or other embodiments, a second system or device may perform one or more, but not necessarily all, of the operations described with respect to both the media data obtainer 102 and the media data analyzer 104 in which the operations performed by the first system or device and the second system or device may not necessarily be the same. Similarly, the delineations and descriptions with respect to the audio media 106, the image media 108, the textual media 110, the audio data 112, the image data 114, the textual data 116, the linked audio data 120, the linked image data 122, and the linked textual data 124 are not meant to be limiting but to help provide understanding of the present disclosure.

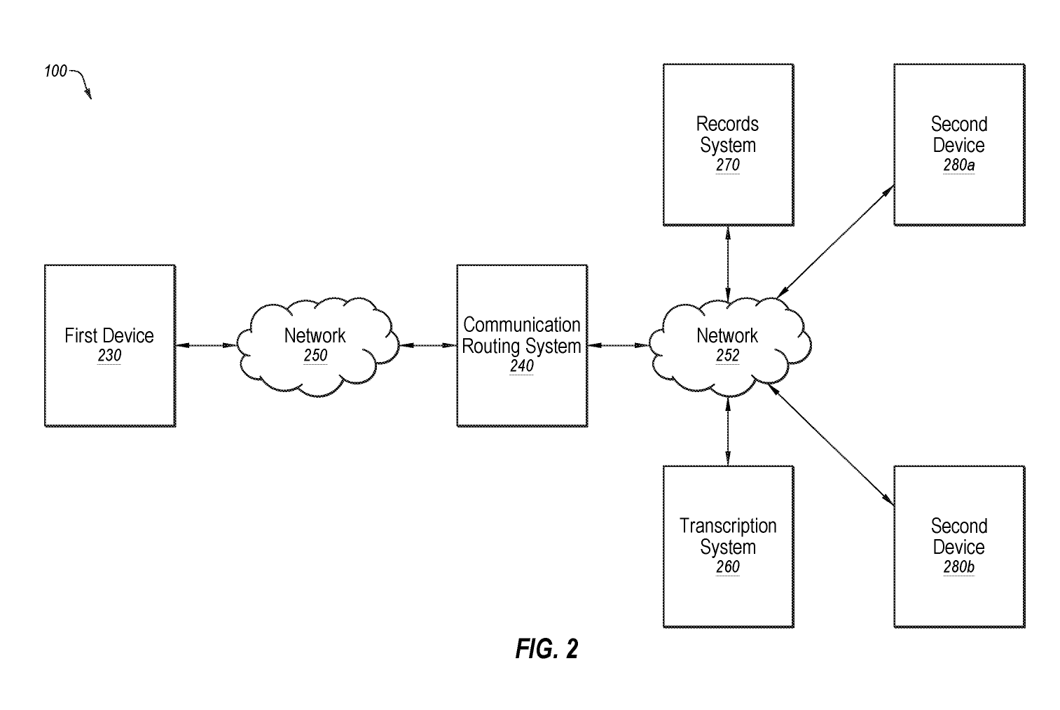

FIG. 2 illustrates an example environment 200 related to linking of media in an example information sharing session between two parties. The environment 200 may be arranged in accordance with at least one embodiment described in the present disclosure. The environment 200 may include a first network 250; a second network 252; a first device 230; second devices 280, including a first second-device 280a and a second second-device 280b; a communication routing system 240; a transcription system 260; and a records system 270.

The first network 250 may be configured to communicatively couple the first device 230 and the communication routing system 240. The second network 252 may be configured to communicatively couple the second devices 280, the communication routing system 240, the transcription system 260, and the records system 270.

In some embodiments, the first and second networks 250 and 252 may each include any network or configuration of networks configured to send and receive communications between devices. In some embodiments, the first and second networks 250 and 252 may each include a conventional type network, a wired or wireless network, and may have numerous different configurations. Furthermore, the first and second networks 250 and 252 may each include a local area network (LAN), a wide area network (WAN) (e.g., the Internet), or other interconnected data paths across which multiple devices and/or entities may communicate.

In some embodiments, the first and second networks 250 and 252 may each include a peer-to-peer network. The first and second networks 250 and 252 may also each be coupled to or may include portions of a telecommunications network for sending data in a variety of different communication protocols. In some embodiments, the first and second networks 250 and 252 may each include Bluetooth® communication networks or cellular communication networks for sending and receiving communications and/or data. The first and second networks 250 and 252 may also each include a mobile data network that may include third-generation (3G), fourth-generation (4G), long-term evolution (LTE), long-term evolution advanced (LTE-A), Voice-over-LTE (“VoLTE”) or any other mobile data network or combination of mobile data networks. Further, the first and second networks 250 and 252 may each include one or more IEEE 602.11 wireless networks. In some embodiments, the first and second networks 250 and 252 may be configured in a similar manner or a different manner. In some embodiments, the first and second networks 250 and 252 may share various portions of one or more networks. For example, each of the first and second networks 250 and 252 may include the Internet or some other network.

The first device 230 may be any electronic or digital device. For example, the first device 230 may include or may be included in a desktop computer, a laptop computer, a smartphone, a mobile phone, a tablet computer, a television set-top box, a smart television, or any other electronic device with a processor. In some embodiments, the first device 230 may include computer-readable-instructions stored on one or more computer-readable media that are configured to be executed by one or more processors in the first device 230 to perform operations described in this disclosure. The first device 230 may be configured to communicate with, receive data from, and direct data to, the communication routing system 240 and/or the second devices 280. During a communication session, audio media, image media, a transcription of the audio, and/or other textual media may be presented by the first device 230.

In some embodiments, the first device 230 may be associated with a first user. The first device 230 may be associated with the first user based on the first device 230 being configured to be used by the first user. In these and other embodiments, the first user may be registered with the communication routing system 240 and the first device 230 may be listed in the registration of the first user. Alternatively or additionally, the first device 230 may be associated with the first user by the first user being the owner of the first device 230 and/or being controlled by the first user.

The second devices 280 may be any electronic or digital devices. For example, the second devices 280 may include, or may be included in, a desktop computer, a laptop computer, a smartphone, a mobile phone, a tablet computer, a television set-top box, a smart television, or any other electronic device with a processor. In some embodiments, the second devices 280 may each include, or be included in, the same, different, or combinations of electronic or digital devices. In some embodiments, the second devices 280 may each include computer-readable instructions stored on one or more computer-readable media that are configured to be executed by one or more processors in the second devices 280 to perform operations described in this disclosure.

The second devices 280 may each be configured to communicate, receive data from and direct data to, the communication routing system 240. Alternatively or additionally, each of the second devices 280 may be configured to, individually or in a group, participate in a communication session with the first device 230 through the communication routing system 240. In some embodiments, the second devices 280 may each be associated with a second user or be configured to be used by a second user. During a communication session, audio media, image media, a transcription of the audio, and/or other textual media may be presented by the second devices 280 for the second users.

In some embodiments, the second users may be health care professionals. In these and other embodiments, health care professionals may be individuals with training or skills to render advice with respect to mental or physical health, including, nurses, nurse practitioners, medical assistants, doctors, physician assistants, counselors, psychiatrists, psychologists, or doulas, among other health care professionals. In these and other embodiments, the first user may be an individual in their home who has a health care need. For example, the first user may be an individual at home who is recovering from a surgery and who has a need for in-home care from a health care professional. Alternatively or additionally, the first user may be an individual at home who has an illness for which in-home care from a health care professional is preferable. Alternatively or additionally, the first user may be an individual at a care facility or some other facility.

In some embodiments, each of the communication routing system 240, the transcription system 260, and the records system 270 may include any configuration of hardware, such as processors, servers, and databases that are networked together and configured to perform one or more tasks. For example, each of the communication routing system 240, the transcription system 260, and the records system 270 may include multiple computing systems, such as multiple servers that each include memory and at least one processor, which are networked together and configured to perform operations as described in this disclosure, among other operations. In some embodiments, each of the communication routing system 240, the transcription system 260, and the records system 270 may include computer-readable instructions on one or more computer-readable media that are configured to be executed by one or more processors in each of the communication routing system 240, the transcription system 260, and the records system 270 to perform operations described in this disclosure. Additionally or alternatively, the communication routing system 240, the transcription system 260, and/or the records system 270 may include at least a portion of the media data obtainer 102 and/or the media data analyzer 104 such as those described above with respect to FIG. 1 such that the communication routing system 240, the transcription system 260, and/or the records system 270 may perform one or more operations of the media data obtainer 102 and/or the media data analyzer 104.

Generally, the communication routing system 240 may be configured to establish and manage communication sessions between the first device 230 and one or more of the second devices 280. The transcription system 260 may be configured to generate and provide transcriptions of audio from communication sessions established by the communication routing system 240.

The records system 270 may be a combination of hardware, including processors, memory, and other hardware configured to store and manage data. In some embodiments, the records system 270 may be configured to generate and/or store data associated with communication sessions such as audio data (e.g., the audio data 112 of FIG. 1), image data (e.g., the image data 114 of FIG. 1), textual data (e.g., the textual data 116 of FIG. 1), linked audio data (e.g., the linked audio data 120 of FIG. 1), linked image data (e.g., the linked image data 122 of FIG. 1), linked textual data (e.g., the linked textual data 124 of FIG. 1), one or more keywords (e.g., the keywords 118 of FIG. 1) and/or follow-up data (e.g., the follow-up data 126 of FIG. 1).

An example of the interaction of the elements illustrated in the environment 200 is now provided. As described below, the elements illustrated in the environment 200 may interact to establish a communication session between the first device 230 and one or more of the second devices 280, to transcribe the communication session, and link media and associated data (including the transcription) that correspond to the communication session in the records system 270.

The first device 230 may send a request for a communication session to the communication routing system 240. The communication routing system 240 may obtain the request from the first device 230. In some embodiments, the request may include an identifier of the first device 230.

Using the identifier of the first device 230, the communication routing system 240 may obtain profile data regarding the first user associated with the first device 230. The profile data may include information about the first user, such as demographic information, including name, age, sex, address, etc., among other demographic data. The profile data may further include health related information about the first user. For example, the health related information may include the height, weight, medical allergies, and current medical conditions, etc., among other health related information. The profile data may further include other information about the first user, such as information that identifies the first user with the records system 270, such as a first user identifier. In some embodiments, the profile data may include transcriptions of conversations between the first user and the second users.

Using the profile data and/or other information about the first user, such as medical data about the first user, the communication routing system 240 may select one or more of the second devices 280 for the communication session with the first device 230. After selecting one or more of the second devices 280, the communication routing system 240 may establish the communication session. Alternatively or additionally, the communication routing system 240 may select one or more of the second devices 280 for the communication session with the first device 230 based on one or more of the second devices 280 being identified in the request from the first device 230.

During a communication session, the first device 230 and the selected one or more second devices 280 may communicate media that may include audio media (e.g., the audio media 106 of FIG. 1), image media (e.g., the image media 108 of FIG. 1) and/or textual data (e.g., the textual data 116 of FIG. 1) between each other. In some embodiments, the communication routing system 240 may be configured to receive the media from the first device 230 and the selected one or more of the second devices 280. Additionally or alternatively, the communication routing system 240 may be configured to route media received from the first device 230 to the selected one or more of the second devices 280. Further, the communication routing system 240 may be configured to route media received from the selected one or more of the second devices 280 to the first device 230.

In these or other embodiments, the communication routing system 240, the first device 230, and the selected one or more of the second devices 280 may be configured in a manner that allows one or more of the second users to control generation of image media by the first device 230. For example, in some embodiments, the first device 230 and the first second device 280a may be participating in a communication session. Additionally, the first device 230, the communication routing system 240, and the first second device 280a may each be configured such that a camera feed of the first device 230 may be routed to the first second device 280a (e.g., via the communication routing system 240) and such that the camera feed may be presented on the first second device 280a. Further, the first second device 280a may be configured to allow a corresponding second user to issue a command on a user interface of the first second device 280a to capture one or more images (e.g., a picture or a video) presented in the camera feed. The first second device 280a may be configured to communicate the command to the first device 230 (e.g., via the communication routing system). In response to receiving the command, the first device 230 may capture the images. In these or other embodiments, the captured image may be communicated to the first second device 280a.

In some embodiments, the communication routing system 240 may route the audio media to the transcription system 260 for generation of transcript data. The transcription system 260 may generate transcript data such that the transcription system 260 may perform one or more operations described above with respect to the media data obtainer 102 of FIG. 1. The transcription system 260 may send the transcript data to the records system 270. The transcript data may also be transmitted to the first device 230 and the selected one or more of the second devices 280 for presentation by the first device 230 and the selected one or more of the second devices 280.

Further explanation of the transcription process and routing is now described. However, it is described in the context of a communication session between the first device 230 and the first second-device 280a for ease of explanation.

As mentioned, the first device 230 and the first second-device 280a may exchange media during a communication session. In some embodiments, the media may include video and audio. For example, the first device 230 may send first audio and first video to the first second-device 280a and the first second-device 280a may send second audio and second video to the first device 230. Alternatively or additionally, the media may include audio but not video.

During the communication session, the media exchanged between the first device 230 and the first second-device 280a may be routed through the communication routing system 240. During the routing of the media between the first device 230 and the first second-device 280a, the communication routing system 240 may be configured to duplicate the audio from the media and provide the duplicated audio to the transcription system 260.

The transcription system 260 may receive the duplicated first audio. The transcription system 260 may generate the first transcript data of the duplicated first audio. The first transcript data may include a transcription of the duplicated first audio.

In some embodiments, the transcription system 260 may generate first transcript data using a machine transcription of the duplicated first audio. In some embodiments, before a machine transcription is made of the duplicated first audio, the duplicated first audio may be listened to and re-voiced by another person. In these and other embodiments, the other person may make corrections to the machine transcription. Additionally or alternatively, in some embodiments, the transcription system 260 may also be configured to generate first transcript timing data with respect to the first transcript data.

The transcription system 260 may provide the first transcript data and the first transcript timing data to the communication routing system 240. The communication routing system 240 may route the first transcript data to the first second-device 280a. The first second-device 280a may present the first transcript data to a user of the first second-device 280a on a display of the first second-device 280a.

The communication routing system 240 and the transcription system 260 may handle the second media from the first second-device 280a in an analogous manner. For example, the communication routing system 240 may generate duplicated second audio of second audio of the second media and the transcription system 260 may generate second transcript data based on the duplicated second audio. The second transcript data may be provided to the first device 230 for presentation of the first user of the first device 230.

In some embodiments, the generation and delivery of the transcript data of the first and second media may both be in substantially real-time or real-time. In these and other embodiments, the first device 230 may present the second transcript data concurrently with the second media data in substantially real-time or real-time. Concurrent presentation of the second transcript data and the second media data in substantially real-time may indicate that when audio is presented, a transcription that corresponds to the presented audio is also presented with a delay of less than 1, 2, 5, 10, or 15 seconds between the transcription and the audio. Alternatively or additionally, the generation and delivery of transcript data of one of the first and second media may be in substantially real-time or real-time and the generation and/or delivery of transcript data of another of the first and second media may not be in real time.

In some embodiments, when a third device, such as the second second-device 280b participates in a communication session between the first device 230 and the first second-device 280a, third transcript data may be generated for third audio generated by the third device. In these and other embodiments, the third transcript data may be provided to the first device 230 and/or the first second-device 280a and the third device may receive the first and/or second transcript data from the first device 230 and the first second-device 280a, respectively.

In some embodiments, the first transcript data and the second transcript data may be combined by interweaving the data segments of the first transcript data and the second transcript data. In these and other embodiments, the data segments of the first transcript data and the second transcript data may be interweaved such that the data segments of the first transcript data and the second transcript data are combined in substantially chronological order.

After generating the first transcript data of the first audio data and the second transcript data of the second audio data, the transcription system 260 may be configured to communicate the first transcript data and the second transcript data to the records system 270. In some embodiments, the transcription system 260 may be configured to combine the first transcript data and the second transcript data prior to communicating the transcript data to the records system 270. Additionally or alternatively, the transcription system 260 may be configured to communicate the first transcript data and the second transcript data separately to the records system 270. In these or other embodiments, the records system 270 may combine the first transcript data and the second transcript data or may leave the first transcript data and the second transcript data separated.

In some embodiments, the communication routing system 240 may be configured to communicate the received audio media, image media, and/or the textual media to the record system 270. In some embodiments, the communication routing system 240 may communicate at least some of the received audio media, image media, and/or textual media during the communication session. For example, in some embodiments, the communication routing system 240 may be configured to duplicate the received audio media, image media, and/or textual media during the communication session and may route the duplicated audio media, image media, and/or textual media to the records system 270 while also routing the received audio media, image media, and/or textual media to the first device 230 and/or the selected one or more second devices 280.

Additionally or alternatively, the communication routing system 240 may communicate at least some of the received audio media, image media, and/or textual media after the communication session. In these or other embodiments, the first device 230 and/or the selected one or more second devices 280 may communicate at least some of the audio media, image media, and/or textual media to the records system 270 during or after the communication system.

In some embodiments, the records system 270 may be configured to generate audio data, image data, textual data, linked audio data, linked image data, linked textual data, one or more keywords, and/or follow-up data such as described above with respect to FIG. 1 regarding the audio data 112, the image data 114, the textual data 116, the linked audio data 120, the linked image data 122, the linked textual data 124, the one or more keywords 118, and/or the follow-up data 126. In these or other embodiments, the communication routing system 240 may be configured to provide the record system 270 with indications related to the sharing and/or capturing of media.

For example, in instances in which the first device 230 captures one or more images in response to a command issued at one of the second devices 280, the communication routing system 240, the first device 230, and/or the corresponding second device 280, may be configured to communicate the occurrence of the command to the record system 270. In these or other embodiments, a capture command and/or a share command related to the capturing of one or more images may be received from the first user at a user interface of the first device 230. In these or other embodiments, the first device 230 may be configured to communicate to the records system 270 that the capture and/or share command was issued. In these or other embodiments, in response to receiving indication of issuance of a particular command, the record system 270 may record issuance of the command, a timing of issuance of the command, and that the command is related to the capturing of one or more images such that image-sharing timing data associated with the one or more images may be generated. In some embodiments, audio-sharing timing data related to the sharing of audio media and/or textual-sharing timing data related to the sharing of textual media may be similarly generated based on similar reporting of corresponding capture and/or sharing commands.

In some embodiments, the record system 270 may be configured to provide at least some of the follow-up data to the first device 230 and/or the selected one or more second devices 280 during the communication session or after the communication session. As described above with respect to FIG. 1, in some instances the follow-up data may provide additional information related to the communication session and/or questions that may be asked during or after the communication session.

Additionally or alternatively, the record system 270 may be configured to provide at least some of the audio data, the image data, the textual data, the linked audio data, the linked image data, and/or the linked textual data to the first device 230 and/or the selected one or more second devices 280. The first device 230 and/or the selected one or more second devices 280 may be configured to present the received audio data, the image data, the textual data, the linked audio data, the linked image data, and the linked textual data to allow for review of the communication session. In some embodiments, the audio data, the image data, the textual data, the linked audio data, the linked image data and the linked textual data generated and configured in the manner described in the present disclosure may help improve review of the communication session by making it easier to experience (e.g., view, listen to, read, etc.) media that may correspond to related subject matter and/or points in time.

Modifications, additions, or omissions may be made to the environment 200 without departing from the scope of the present disclosure. For example, in some embodiments, the transcription system 260 may be part of the communication routing system 240. Alternatively or additionally, the transcription system 260, the communication routing system 240, and the records system 270 may all be part of one system.

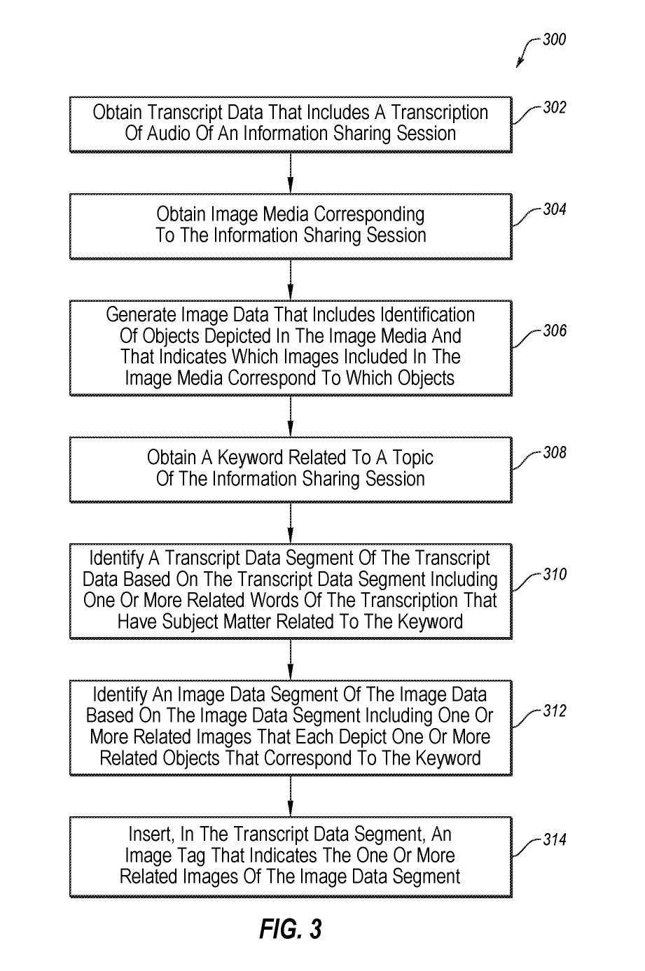

FIG. 3 is a flowchart of an example method 300 of linking media data related to an information sharing session. The method 300 may be arranged in accordance with at least one embodiment described in the present disclosure. The method 300 may be performed, in whole or in part, in some embodiments by a system or combinations of components in a system or environment as described in the present disclosure. For example, the method 300 may be performed, in whole or in part, by environment 100, environment 200 and/or the system 600 of FIGS. 1, 2, and 6, respectively. In these and other embodiments, some or all of the operations of the method 300 may be performed based on the execution of instructions stored on one or more non-transitory computer-readable media. Although illustrated as discrete blocks, various blocks may be divided into additional blocks, combined into fewer blocks, or eliminated, depending on the particular implementation.

The method 300 may begin at block 302, where transcript data of audio of an information sharing session may be obtained. In some embodiments, the information sharing session may be a communication session and the audio may include first device audio sent from a first device to a second device during the communication session. In these or other embodiments, the audio may include second device audio sent from the second device to the first device during the communication session. The audio media 106 described above with respect to FIG. 1 may be an example of the audio that may be obtained. The transcript data described as being included in the audio data 112 of FIG. 1 may be an example of the transcript data that is obtained. In some embodiments, the obtaining of the transcript data may include generating the transcript data. In these or other embodiments, the obtaining of the transcript data may include receiving the transcript data. Additionally or alternatively, the obtaining of the transcript data may include directing the generation of the transcript data by another system and receiving the transcript data generated in response to the direction.

At block 304, image media corresponding to the information sharing session may be obtained. In some embodiments, the image media may be communicated between the first device and the second device during the communication session. The image media 108 described above with respect to FIG. 1 may be an example of the image media that may be obtained.

At block 306, image data that includes identification of objects depicted in the image media and that indicates which images included in the image media correspond to which objects may be generated. The image data 114 of FIG. 1 may be an example of the image data that is generated.

At block 308, a keyword related to a topic of the information sharing session may be obtained. A keyword 118 of FIG. 1 may be an example of the keyword that may be obtained. Additionally, the keyword may be obtained according to as described above with respect to FIG. 1 in some embodiments.

At block 310, a transcript data segment of the transcript data may be identified. In some embodiments, the transcript data segment may be identified based on the transcript data segment including one or more related words of the transcription that have subject matter related to the keyword. The transcript data segment may be identified according to as described above with respect to FIG. 1 in some embodiments.

At block 312, an image data segment of the image data may be identified. In some embodiments, the image data segment may be identified based on the image data segment including one or more related images that each depict one or more related objects that correspond to the keyword. The image data segment may be identified according to as described above with respect to FIG. 1 in some embodiments.

At block 314, in response to the transcript data segment and the image data segment both corresponding to the keyword, an image tag that indicates the one or more related images of the image data segment may be inserted in the transcript data segment. The insertion of the image tag in the transcript data segment may create linked transcript data that may be included in linked audio data such as described above with respect to FIG. 1. The image tags described above with respect to FIG. 1 may be examples of the image tag that may be inserted at block 314.

One skilled in the art will appreciate that, for these processes, operations, and methods, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

For example, in some embodiments, the method 300 may include one or more operations related to establishing the communication session between the first device and the second device such that the first device audio is sent from the first device to the second device and such that the second device audio is sent from the second device to the first device during the communication session. In these or other embodiments, the method 300 may include one or more operations related to receiving the first device audio as the first device audio is routed to the second device and/or receiving the second device audio as the second device audio is routed to the first device.

Additionally or alternatively, in some embodiments, the method 300 may include one or more operations related to inserting, in the image data segment, a transcript tag that indicates the one or more related words of the transcript data segment. The transcript tags described above with respect to FIG. 1 may be examples of the transcript tag.

In these or other embodiments, the method 300 may include one or more operations related to obtaining textual data that is related to textual media that is communicated during the information sharing session and identifying a textual data segment of the textual data based on the textual data segment including one or more related portions of the textual media that have subject matter related to the keyword. In these or other embodiments, in response to the transcript data segment and the textual data segment both corresponding to the keyword, the method 300 may include one or more operations related to inserting, in the transcript data segment, a textual tag that indicates the one or more related portions of the textual data segment. The textual tags described above with respect to FIG. 1 may be examples of the textual tag.

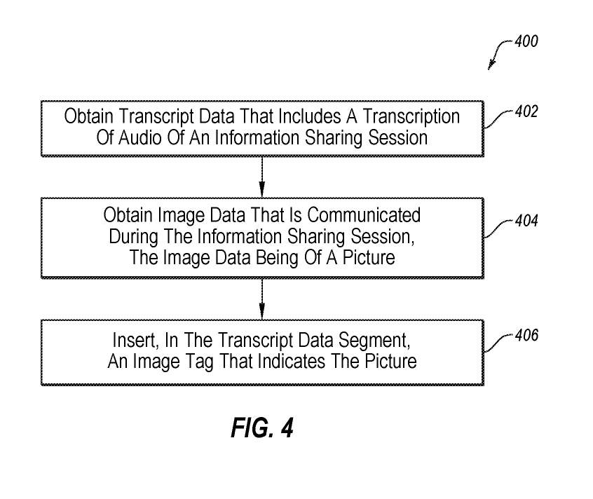

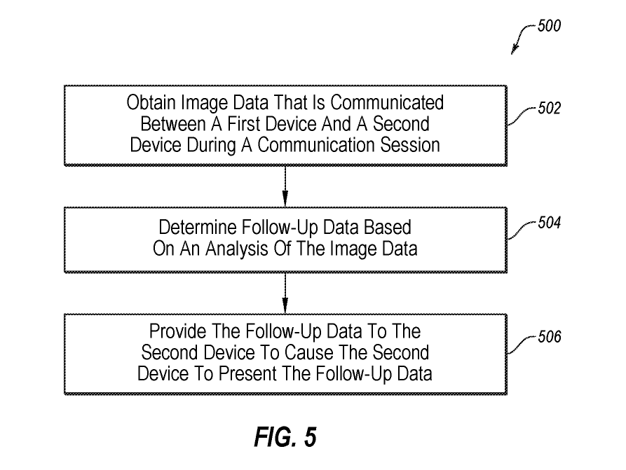

Additionally or alternatively, the textual tag may be inserted in the image data segment in response to the image data segment and the textual data segment both corresponding to the keyword. In these or other embodiments, the transcript tag and/or the image tag may be inserted in the textual data segment in response to the transcript data segment, the textual data segment, and the image data segment all corresponding to the keyword. In these or other embodiments, one or more of the tags may be inserted in one or more of the data segments in response to the data segments each having timing data that corresponds to points in time during the information sharing session that are within a particular timeframe with respect to each other. In these or other embodiments, the method 300 may include one or more operations described below with respect to the methods 400 and 500 of FIGS. 4 and 5, respectively.

FIG. 4 is a flowchart of another example method 400 of linking media data related to an information sharing session. The method 400 may be arranged in accordance with at least one embodiment described in the present disclosure. The method 400 may be performed, in whole or in part, in some embodiments by a system or combinations of components in a system or environment as described in the present disclosure. For example, the method 400 may be performed, in whole or in part, by environment 100, environment 200 and/or the system 600 of FIGS. 1, 2, and 6, respectively. In these and other embodiments, some or all of the operations of the method 400 may be performed based on the execution of instructions stored on one or more non-transitory computer-readable media. Although illustrated as discrete blocks, various blocks may be divided into additional blocks, combined into fewer blocks, or eliminated, depending on the particular implementation.

The method 400 may begin at block 402, where transcript data of audio of an information sharing session may be obtained. In some embodiments, the information sharing session may be a communication session and the audio may include first device audio sent from a first device to a second device during the communication session. In these or other embodiments, the audio may include second device audio sent from the second device to the first device during the communication session. The audio media 106 described above with respect to FIG. 1 may be an example of the audio that may be obtained. The transcript data described as being included in the audio data 112 of FIG. 1 may be an example of the transcript data that is obtained. In some embodiments, the obtaining of the transcript data may include generating the transcript data. In these or other embodiments, the obtaining of the transcript data may include receiving the transcript data. Additionally or alternatively, the obtaining of the transcript data may include directing the generation of the transcript data by another system and receiving the transcript data generated in response to the direction.

At block 404, image data that is communicated during the information sharing session may be obtained. In some embodiments, the image data may include an image file that may be of one or more images such as a picture or a video. The image data 114 of FIG. 1 may be an example of the image data that may be obtained.

At block 406, an image tag that indicates the one or more images may be inserted in a transcript data segment of the transcript data. In some embodiments, the image tag may be inserted in response to the image data and the transcript data segment each having timing data that corresponds to points in time during the communication session that are within a particular timeframe with respect to each other. In some embodiments, the image data and the transcript data segment may be determined as each having timing data that corresponds to points in time during the communication session that are within the particular timeframe with respect to each other based on timestamps such as discussed above with respect to FIG. 1. In these or other embodiments, the timestamps may be obtained in one or more of the manners described above with respect to FIG. 1.

One skilled in the art will appreciate that, for these processes, operations, and methods, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

For example, in some embodiments, the method 400 may include one or more operations related to establishing the communication session between the first device and the second device such that the first device audio is sent from the first device to the second device and such that the second device audio is sent from the second device to the first device during the communication session. In these or other embodiments, the method 400 may include one or more operations related to receiving the first device audio as the first device audio is routed to the second device and/or receiving the second device audio as the second device audio is routed to the first device.

Additionally or alternatively, in some embodiments, the method 400 may include one or more operations related to inserting, in the image data, a transcript tag that indicates the one or more related words of the transcript data segment. In these or other embodiments, the transcript tag may be inserted in the image data in response to the image data and the transcript data segment each having timing data that corresponds to points in time during the communication session that are within the particular timeframe with respect to each other. The transcript tags described above with respect to FIG. 1 may be examples of the transcript tag. In these or other embodiments, the method 400 may include one or more operations described with respect to the methods 300 and 500 of FIGS. 3 and 5, respectively.

FIG. 5 is a flowchart of an example method 500 of determining follow-up data related to an information sharing session. The method 500 may be arranged in accordance with at least one embodiment described in the present disclosure. The method 500 may be performed, in whole or in part, in some embodiments by a system or combinations of components in a system or environment as described in the present disclosure. For example, the method 500 may be performed, in whole or in part, by environment 100, environment 200 and/or the system 600 of FIGS. 1, 2, and 6, respectively. In these and other embodiments, some or all of the operations of the method 500 may be performed based on the execution of instructions stored on one or more non-transitory computer-readable media. Although illustrated as discrete blocks, various blocks may be divided into additional blocks, combined into fewer blocks, or eliminated, depending on the particular implementation.

The method 500 may begin at block 502, where image data that is communicated between a first device and a second device during a communication session may be obtained. In some embodiments, the image data may include an image file that may be of one or more images such as a picture or a video. The image data 114 of FIG. 1 may be an example of the image data that may be obtained.

At block 504, follow-up data related to the communication session may be determined based on an analysis of the image data. In some embodiments, the follow-up data may include additional information and/or questions related to subject matter discussed during the communication session. In some embodiments, the follow-up data 126 of FIG. 1 may be an example of the follow-up data that may be determined at block 504. Additionally or alternatively, the follow-up data determined at block 504 may be determined according to one or more operations described above with respect to determining the follow-up data 126 of FIG. 1.

At block 506, the follow-up data may be provided to the second device to cause the second device to present the follow-up data. In these or other embodiments, the follow-up data may be provided to the first device to cause the first device to present the follow-up data. In some embodiments, the follow-up data may be provided during the communication session such that the follow-up data may be presented during the communication session. Additionally or alternatively, the follow-up data may be provided after the communication session.

One skilled in the art will appreciate that, for these processes, operations, and methods, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments. For example, in some embodiments, the method 500 may include one or more operations related to the one or more images being captured by the first device in response to a command performed by a user on a user interface of the second device. For example, in some embodiments, the method 500 may include one or more operations described with respect to the methods 300 and 400 of FIGS. 3 and 4, respectively.

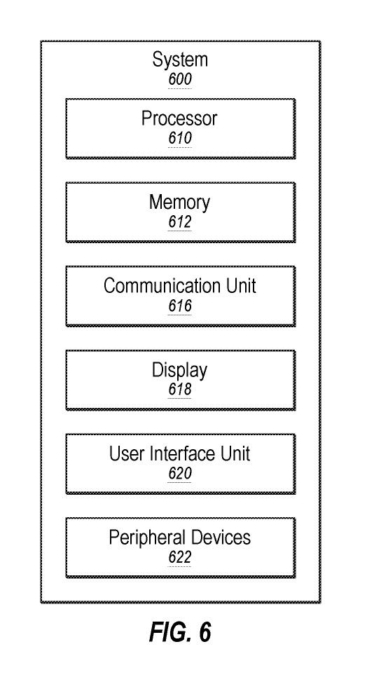

FIG. 6 illustrates an example computing system 600 that may be used to link media data and/or to generate follow-up data related to an information sharing session. The system 600 may be arranged in accordance with at least one embodiment described in the present disclosure. The system 600 may include a processor 610, memory 612, a communication unit 616, a display 618, a user interface unit 620, and a peripheral device 622, which all may be communicatively coupled. In some embodiments, the system 600 may be part of any of the systems or devices described in this disclosure.

For example, the system 600 may be part of the first device 230 of FIG. 2 and may be configured to perform one or more of the tasks described above with respect to the first device 230. As another example, the system 600 may be part of the second devices 280 of FIG. 2 and may be configured to perform one or more of the tasks described above with respect to the second devices 280. As another example, the system 600 may be part of the transcription system 260 of FIG. 2 and may be configured to perform one or more of the tasks described above with respect to the transcription system 260. As another example, the system 600 may be part of the records system 270 of FIG. 2 and may be configured to perform one or more of the tasks described above with respect to the records system 270. As another example, the system 600 may be part of the communication routing system 240 of FIG. 2 and may be configured to perform one or more of the tasks described above with respect to the communication routing system 240.

Generally, the processor 610 may include any suitable special-purpose or general-purpose computer, computing entity, or processing device including various computer hardware or software modules and may be configured to execute instructions stored on any applicable computer-readable storage media. For example, the processor 610 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data.

Although illustrated as a single processor in FIG. 6, it is understood that the processor 610 may include any number of processors distributed across any number of networks or physical locations that are configured to perform individually or collectively any number of operations described herein. In some embodiments, the processor 610 may interpret and/or execute program instructions and/or process data stored in the memory 612. In some embodiments, the processor 610 may execute the program instructions stored in the memory 612.

For example, in some embodiments, the media data obtainer 102 and/or the media data analyzer 104 of FIG. 1 may be included in the memory 612 as program instructions. The processor 610 may execute the corresponding program instructions from the memory such that the system 600 may perform or direct the performance of the operations associated with the media data obtainer 102 and/or the media data analyzer 104 as directed by the instructions. In these and other embodiments, instructions may be used to perform one or more of the methods 300, 400, and 500 of FIGS. 3, 4, and 5 respectively.

The memory 612 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 610. By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 610 to perform a certain operation or group of operations as described in this disclosure. In these and other embodiments, the term “non-transitory” as explained in the present disclosure should be construed to exclude only those types of transitory media that were found to fall outside the scope of patentable subject matter in the Federal Circuit decision of In re Nuijten, 500 F.3d 1346 (Fed. Cir. 2007). Combinations of the above may also be included within the scope of computer-readable media.

The communication unit 616 may include any component, device, system, or combination thereof that is configured to transmit or receive information over a network. In some embodiments, the communication unit 616 may communicate with other devices at other locations, the same location, or even other components within the same system. For example, the communication unit 616 may include a modem, a network card (wireless or wired), an infrared communication device, a wireless communication device (such as an antenna), and/or chipset (such as a Bluetooth device, an 802.6 device (e.g., Metropolitan Area Network (MAN)), a WiFi device, a WiMax device, cellular communication facilities, etc.), and/or the like. The communication unit 616 may permit data to be exchanged with a network and/or any other devices or systems described in the present disclosure. For example, when the system 600 is included in the first device 230 of FIG. 2, the communication unit 616 may allow the first device 230 to communicate with the communication routing system 240.

The display 618 may be configured as one or more displays, like an LCD, LED, or other type of display. The display 618 may be configured to present video, text captions, user interfaces, and other data as directed by the processor 610. For example, when the system 600 is included in the first device 230 of FIG. 2, the display 618 may be configured to present second video from a second device and a transcript of second audio from the second device.

The user interface unit 620 may include any device to allow a user to interface with the system 600. For example, the user interface unit 620 may include a mouse, a track pad, a keyboard, buttons, and/or a touchscreen, among other devices. The user interface unit 620 may receive input from a user and provide the input to the processor 610.

The peripheral devices 622 may include one or more devices. For example, the peripheral devices may include a microphone, an imager, and/or a speaker, among other peripheral devices. In these and other embodiments, the microphone may be configured to capture audio. The imager may be configured to capture digital images. The digital images may be captured in a manner to produce video or image data. In some embodiments, the speaker may broadcast audio received by the system 600 or otherwise generated by the system 600. Modifications, additions, or omissions may be made to the system 600 without departing from the scope of the present disclosure. For example, the system 600 may not include one or more of: the display 618, the user interface unit 620, and peripheral device 622.

Modifications, additions, or omissions may be made to the system 600 without departing from the scope of the present disclosure. For example, in some embodiments, the system 600 may include any number of other components that may not be explicitly illustrated or described. Further, depending on certain implementations, the system 600 may not include one or more of the components illustrated and described.

In some embodiments, the different components, modules, engines, and services described herein may be implemented as objects or processes that execute on a computing system (e.g., as separate threads). While some of the systems and methods described herein are generally described as being implemented in software (stored on and/or executed by general purpose hardware), specific hardware implementations or a combination of software and specific hardware implementations are also possible and contemplated.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A computer-implemented method to link media data related to a communication session, the method comprising:
obtaining transcript data that includes a transcription of audio of a communication session;
receiving image media that is communicated between a first device and a second device during the communication session;
generating image data that includes identification of objects depicted in the image media and that indicates which images included in the image media correspond to which objects;
obtaining a keyword related to a topic of the communication session;
identifying a transcript data segment of the transcript data based on the transcript data segment including a related word of the transcription that has subject matter related to the keyword;
identifying an image data segment of the image data based on the image data segment including a related image that depicts a related object that corresponds to the keyword; and
in response to the transcript data segment and the image data segment both corresponding to the keyword:
inserting, in the transcript data segment, an image tag that indicates the related image of the image data segment; and
inserting, in the image data segment, a transcript tag that indicates the related word of the transcript data segment.

2. The method of claim 1, wherein the image tag includes a selectable image link that indicates the related image of the image data segment by providing access to the related image of the image data segment in response to selection of the selectable image link.

3. The method of claim 1, further comprising inserting the image tag in the transcript data segment and inserting the transcript tag in the image data segment in response to the image data segment and the transcript data segment each having timing data that corresponds to points in time during the communication session that are within a particular timeframe with respect to each other.

4. The method of claim 1, wherein the transcript tag includes a selectable transcript link that indicates the related word of the transcript data segment by providing access to the transcript data segment in response to selection of the selectable transcript link.

5. The method of claim 1, wherein generating the image data includes:
performing image recognition with respect to the image media to identify the objects depicted in the image media; and
associating the objects with the images from which the objects were identified.

6. The method of claim 1, further comprising obtaining the keyword based on a profile of a participant in the communication session.

7. The method of claim 1, further comprising:
obtaining textual data related to textual media that is communicated between the first device and the second device during the communication session;
identifying a textual data segment of the textual data based on the textual data segment including a related portion of the textual media that have subject matter related to the keyword; and
in response to the transcript data segment and the textual data segment both corresponding to the keyword, inserting, in the transcript data segment, a textual tag that indicates the related portion of the textual data segment.

8. One or more non-transitory computer-readable media configured to store instructions that in response to being executed by one or more processors cause one or more systems to perform the method of claim 1.

9. A computer-implemented method to link media data related to an information sharing session, the method comprising:
obtaining transcript data that includes a transcription of audio of an information sharing session;
obtaining image media corresponding to the information sharing session;
generating image data that includes identification of objects depicted in the image media and that indicates which images included in the image media correspond to which objects;
obtaining a keyword related to a topic of the information sharing session;
identifying a transcript data segment of the transcript data based on the transcript data segment including a related word of the transcription that have subject matter related to the keyword;
identifying an image data segment of the image data based on the image data segment including a related image that depicts a related object that corresponds to the keyword; and
in response to the transcript data segment and the image data segment both corresponding to the keyword, inserting, in the transcript data segment, an image tag that indicates the related image of the image data segment.

10. The method of claim 9, further comprising, in response to the transcript data segment and the image data segment both corresponding to the keyword, inserting, in the image data segment, a transcript link that indicates the related word of the transcript data segment.

11. The method of claim 9, wherein the image tag includes a selectable image link that indicates the related image of the image data segment by providing access to the related image of the image data segment in response to selection of the selectable image link.

12. The method of claim 9, further comprising inserting the image tag in the transcript data segment in response to the image data segment and the transcript data segment each having timing data that corresponds to points in time during the information sharing session that are within a particular timeframe with respect to each other.

13. The method of claim 9, wherein:
the information sharing session includes a communication session between a first person via a first device and a second person via a second device in which first device audio is sent from the first device to the second device and second device audio is sent from the second device to the first device during the communication session;
the audio of the information sharing session includes the first device audio and the second device audio; and
obtaining the transcript data includes generating the transcript data from the first device audio and the second device audio.

14. One or more non-transitory computer-readable media configured to store instructions that in response to being executed by one or more processors cause one or more systems to perform the method of claim 9.

15. A computer-implemented method to link media data related to a communication session, the method comprising:
obtaining transcript data that includes a transcription of audio of a communication session;
receiving image media that is communicated between a first device and a second device during the communication session;
generating image data that includes identification of objects depicted in the image media and that indicates which images included in the image media correspond to which objects;
obtaining a keyword related to a topic of the communication session;
identifying a transcript data segment of the transcript data based on the transcript data segment including a related word of the transcription that has subject matter related to the keyword;
identifying an image data segment of the image data based on the image data segment including a related image that depicts a related object that corresponds to the keyword; and
in response to the transcript data segment and the image data segment both corresponding to the keyword, inserting, in the transcript data segment, an image tag that indicates the related image of the image data segment.

16. The method of claim 15, wherein the image tag includes a selectable image link that indicates the related image of the image data segment by providing access to the related image of the image data segment in response to selection of the selectable image link.

17. The method of claim 15, wherein generating the image data includes:
performing image recognition with respect to the image media to identify the objects depicted in the image media; and
associating the objects with the images from which the objects were identified.

18. The method of claim 15, further comprising:
obtaining textual data related to textual media that is communicated between the first device and the second device during the communication session;
identifying a textual data segment of the textual data based on the textual data segment including a related portion of the textual media that has subject matter related to the keyword; and
in response to the transcript data segment and the textual data segment both corresponding to the keyword, inserting, in the transcript data segment, a textual tag that indicates the related portion of the textual data segment.

19. The method of claim 15 further comprising inserting the image tag in the transcript data segment in response to the image data segment and the transcript data segment each having timing data that corresponds to points in time during the communication session that are within a particular timeframe with respect to each other.

20. One or more non-transitory computer-readable media configured to store instructions that in response to being executed by one or more processors cause one or more systems to perform the method of claim 15.
